(19) United States
(12) Patent Application Publication
(10) Pub. No.: US 2009/0246806 A1
(43) Pub. Date: Oct. 1, 2009

(54) COCCIDIOIDES ANTIGEN IMMUNOASSAY

(76) Inventor: L. JOSEPH WHEAT, Indianapolis, IN (US)

Correspondence Address: Brinks Hofer Gilson & Lione/Ann Arbor, 524 South Main Street, Suite 200, Ann Arbor, MI, 48104, US

(21) Appl. No.: 12/415,443

(22) Filed: Mar. 31, 2009

Related U.S. Application Data
(60) Provisional application No. 61/041,460, filed on Apr. 1, 2008.

(51) Int. Cl. G01N 33/569

(52) U.S. Cl. 435/7.31

(57) ABSTRACT

The present disclosure relates to methods for detecting a Coccidioides in a physiological specimen. Methods, materials and kits relating to the detection of Coccidioides antigen by improved EIA methods are described.

### RELATED APPLICATIONS

This application claims the benefit of U.S. Patent Application No. 61/041,460, filed Apr. 1, 2009 which is incorporated by reference herein in its entirety. This application incorporates by reference both U.S. patent application Ser. No. 11/491,647, filed Jul. 24, 2006 and U.S. provisional patent application Ser. No. 60/702,653, filed Jul. 25, 2005, herein in their entirety.

### FIELD

The present invention relates to the field of medical diagnostics, particularly with respect to fungal pathogens. More particularly, the present invention relates to immunoassay detection of fungal antigens, including Coccidioides.

### BACKGROUND

Coccidioidomycosis is acquired by the inhalation of the mycelia, which transforms to the spherule in tissues. The genus Coccidioides includes two species, immitis and posadasii, which for convenience are included in the designation Coccidioides. Coccidioides is a pathogenic dimorphic fungus that grows as mycelia in nature and spherule in humans and animals. Inhalation of airborne mycelia results in a morphological transformation to the spherule form, which may cause pulmonary infection and occasional progressive disease, particularly in immunosuppressed patients. Coccidioidomycosis is highly endemic in the Southwestern regions of the United States. Most infections are not clinically recognized, and most symptomatic cases in otherwise healthy individuals are mild and are resolved without therapy. Among healthy individuals who are symptomatic, most present with acute pneumonia. Patients with underlying diseases may develop progressive coccidioidomycosis, which often is fatal. Coccidioides is on the list of pathogens considered by the United States to be potential agents of bioterrorism.

In endemic regions, coccidioidomycosis is a common cause of pneumonia and systemic illness. In a report of ambulatory patients with community acquired pneumonia from Arizona, 29% proved to have coccidioidomycosis. Following heavy exposure or in patients who are immunosuppressed, have diabetes or history of extensive tobacco use pneumonia may be severe. Severe illness may be prevented by early treatment of acute coccidioidal pneumonia. The diagnosis of coccidioidomycosis can sometimes be difficult, particularly in immunosuppressed patients with acute, severe disease. Delayed diagnosis is one important reason for a poor outcome in coccidioidomycosis. Mortality has ranged from 40 to 73% in immunocompromised patients. The clinical and other laboratory findings were not specific, but rather consistent with other etiologies including bacterial infection manifested as a systemic inflammatory response syndrome with progressive cardiorespiratory failure. Mortality was 73% in that report, in part because of delay in diagnosis due to the slow growth of the organism in culture.

Coccidioidomycosis has become the leading cause of community acquired pneumonia in endemic areas (Valdivia CID 2006). Of hospitalized patients with coccidioidomycosis, the duration of stay averages six days at a cost of $26,000 and mortality of 6% (Chu CID 2002). Risk factors for a fatal course include heavy exposure, old age, immunosuppression and African-American race (Rosenstein CID 2001). Deficiencies in the currently available tests include false-negative results for cytology, serology and culture and slow results for serology and culture. Serology may be falsely negative early in the course of the infection and in immunosuppressed individuals.

The diagnosis of coccidioidomycosis in humans is often suggested by results of a careful clinical evaluation and radiologic studies, but laboratory tests are necessary to confirm the diagnosis. Isolation of the organism from blood or tissue provides a definitive diagnosis. Tests routinely used for diagnosis of coccidioidomycosis include cytology, histopathology, culture and serology each having notable limitations. Cytology or histopathology may provide a rapid diagnosis, but usually require an invasive procedure and may be negative in up one third of cases. In an analysis of cases of severe coccidioidomycosis, cultures were positive in only 6% of pulmonary cases and 39% of disseminated cases. Furthermore, cultures for Coccidioides can require more than a week to be reported positive. Antibody testing may be negative early the course of illness, and in immunosuppressed patients. The most widely available tests are the enzyme immunoassay, immunodiffusion assay and complement fixation test, which are traditionally performed with antigens prepared from the mycelia. The enzyme immunoassay is more sensitive but less specific than immunodiffusion or complement fixation, and has been used to detect IgM and IgG antibodies to mycelia antigens as a screening test for coccidioidomycosis. Because the enzyme immunoassay are not specific for coccidioidomycosis, if the enzyme immunoassay is positive, it must be confirmed by demonstrating positivity by immunodiffusion or complement fixation.

Coccidioides antigen detection using a competitive enzyme immunoassay has been reported in coccidioidomycosis but this technology was not refined for clinical use. We have observed that the Histoplasma antigen test was positive in patients with coccidioidomycosis. Most patients with positive antigen results in that report had positive cultures for Coccidioides and none had positive cultures for Histoplasma. This result shows that the Histoplasma antigen test is able to detect both Histoplasma and Coccidioides antigen. These findings support the potential for use of antigen detection in diagnosis of coccidioidomycosis. However, because the Histoplasma antigen test also detects Histoplasma and Blastomyces antigen, a positive result in the Histoplasma antigen test does not, by itself, provide for a diagnosis of coccidioidomycosis.

Enzyme linked immunosorbant assay (ELISA) is a sensitive analytical technique used for determination of the concentration of certain antigens and antibodies. ELISA is a useful tool in disease diagnosis, including detection of fungal infection such as coccidioidomycosis. ELISA is typically performed using a polystyrene microtiter detection plate with a capture antibody or antigen immobilized onto the surface of the wells of the microtiter plate. ELISA protocols can be designed in a heterogeneous format or a homogeneous format. A standard ELISA using a heterogeneous format involves a series of incubations of a surface with a reagent contained in a physiological buffer separated by washes to remove material that did not bind to the surface. In contrast, a homogeneous format ELISA includes no requirement for wash steps between incubations of the surface with the various reagents, such as is the case with the commercially-available CEDIA® (Boehringer Mannheim Gmbh) and EMIT® (Behring Diagnostics Inc.) technologies that are currently in use with other immunoassays. The enzyme-linked immunoassay system can be configured to detect one or more antigens in an analyte sample.

However, the reliability of these tests can be hampered by false-positive or false-negative reactions, particularly in individuals unknowingly carrying human anti-animal antibodies (HARA). A false-positive result may occur when the analyte contains HARA because the HARA may bind to the capture antibody even in the absence of Coccidioides antigen. A rabbit anti-Coccidioides detector antibody then could be bound by the HARA leading to detection of a signal even in the absence of Coccidioides antigen. Potentially the HARA could bind to the capture antibody in such a way as to interfere with antigen binding, and may result in a false-negative result. Human anti-animal antibodies typically go undetected in patients, often resulting in false-positive or false-negative readings from ELISA tests for pathogenic antigens. False-positive readings can result in unnecessary medical intervention, while false-negative readings can lead to mis-diagnosis or failure to administer appropriate medical care. Human anti-animal antibodies are more likely to be present in patients after the administration of a pharmaceutical or diagnostic agent derived from an animal source. For example, Wheat et al. identified false-positive test results in individuals without histoplasmosis in 2002 (as described in Wheat L J, Garringer T, Brizendine E, and Connolly P., “Diagnosis of histoplasmosis by antigen detection based upon experience at the Histoplasmosis reference laboratory,” Diagn Microbiol Infect Dis 2002; 43:29-37; Wheat L J. Current diagnosis of histoplasmosis. Trends Microbiol 2003; 11:488-94), incorporated herein by reference in its entirety. One cause for false-positive results was identified in organ allograft recipients who received Thymoglobulin®, as described by Wheat L J, Connolly P, Durkin M et al. False-positive Histoplasma antigenemia caused by antithymocyte globulin antibodies. Transpl Infect Dis 2004; 6:23-7. False-positive antigenemia correlated highly with the presence of HARA. This type of interference activity has also been recognized in assays using murine antibodies, for example as described in Kricka L J., “Human anti-animal antibody interferences in immunological assays,” Clin Chem 45:942-56 (1999).

Accordingly, there is a need for immunoassay tests to identify pathogens, such as Coccidioides, using animal-derived capture and/or detection antibodies in the presence of a human anti-animal antibody, including HARA. Diagnostic tests for fungal antigens generally, and for Coccidioides antigen in particular, are needed that have a reduced or low level of false-positives or false-negatives. In particular, immunoassay tests for detection of a Coccidioides antigen in the presence of HARA are needed.

### SUMMARY

The present disclosure relates to enzyme-linked immunoassay (“ELISA”) kits, procedures and diagnostic methods for identifying Coccidioides antigen. The present disclosure provides for ELISA assay, kits, procedures and methods that provide a desirably reduced incidence of false-positives and/or false-negatives when detecting the antigen. The present disclosure provides for an assay that is more sensitive and specific than prior assays for detecting Coccidioides antigens in specimens from patients with coccidioidomycosis. This sensitivity and specificity allows for a more accurate diagnosis of a patient having coccidioidomycosis. The ability to identify the correct pathogen is important because it allows physicians to choose the most effective treatment based on the identification of the pathogen. Preferred ELISA assays according to the present disclosure may have reduced incidence of false-positives or negatives caused by the human anti-animal antibodies, including HARA that may be present in a patient sample. The preferred immunoassays are preferably configured as sandwich (two-site) ELISA immunoassays performed by contacting a sample with a capture antibody bound to an antigen binding surface, e.g., a microtiter plate and contacting the bound antigen to a suitable detector antibody.

An embodiment according to the present disclosure is an ELISA assay in which there is both a capture antibody and a detector antibody, which are directed to Coccidioides antigens. In one embodiment both the capture antibody and the detector antibodies are polyclonal rabbit anti-Coccidioides IgG. In another embodiment according to the present disclosure the capture antibody comprises an unmodified polyclonal rabbit anti-Coccidioides IgG, and the detector antibody is a modified polyclonal rabbit anti-Coccidioides IgG that does not comprise an F_{c} antibody portion, e.g. an F(ab′)₂ or F(ab) fragment of the antibody. Polyclonal rabbit anti-Coccidioides antibodies according to the present disclosure may be generated against Coccidioides mould or against galactomannan which is purified from Coccidioides mould. In some embodiments the capture antibody or the detector antibody is pooled from two or more immunized rabbits.

The detector antibody is adapted for detection by a suitable method, such as radiologic or optical detection. The detector antibody may be adapted to bind to a reporting molecule to detect the presence of the detector antibody attached to a surface-bound antigen. Antigen-binding results may be classified as positive or negative by comparison with a suitable control, either a positive or negative control. The detected signal in some embodiments according to the present disclosure is measured by optical density (OD). The optical density measured in an assay according to the present disclosure can be divided by the cutoff optical density to obtain results reported in assay units (above 1 U being positive for the presence of a particular antigen). Preferably, the results are reported quantitatively in units of mass of antigen per unit volume of specimen. Quantitative reporting of antigen levels can be obtained by comparison of assay results with a calibration curve derived by using samples of known antigen concentration, referred to as “calibrators”.

The immunoassays preferably include one or more refinements as disclosed herein, relating to uniformity in blocking agents, detector antibody configurations, compositions comprising the detector antibody, and reporting of antigen binding data in a quantitative format.

### BRIEF DESCRIPTION OF THE DRAWINGS

FIG. 1 is a schematic of a sandwich (two site) immunoassay.

FIG. 2 is a graph showing the results of detection of antigens in urine specimens from patients with coccidioidomycosis, histoplasmosis and healthy subjects in a Coccidioides antigen immunoassay.

FIG. 3A is a graph showing fractions from a DEAE column containing Coccidioides galactomannan. This material was prepared by concanavalin A chromatography (con A) of an ethanol precipitate of mould culture supernatant.

FIG. 3B is an SDS PAGE profile corresponding to the data in FIG. 3A. The first lane contains lane markers and the other lanes contain DEAE fractions. Lanes 8-12 contains fractions 24-32, showing close correlation with the OD₂₁₄ carbohydrate peak.

FIG. 4 is a calibration curve based on dilutions of a calibration standard comprising known amounts of a Coccidioides and Histoplasma mould galactomannan, showing detection of purified galactomannan in MVista® Coccidioides or Histoplasma antigen assays.

FIG. 5 is a graph showing three lots of calibration standards prepared from purified Coccidioides mould galactomannan.

FIG. 6 shows calibration curves of purified galactomannan and urine calibrators. A urine specimen containing Coccidioides galactomannan was diluted in Starting Block™ to prepare the urine calibrators, which were tested in the Coccidioides antigen EIA, and assigned a galactomannan concentration by comparison to purified galactomannan.

FIG. 7 is a graph showing the results of detection of antigens in patient specimens from patients with coccidioidomycosis, other mycoses or non-fungal infections and healthy subjects in a Coccidioides antigen immunoassay.

### DETAILED DESCRIPTION

The following definitions are offered to lend clarity to this writing, wherein to the extent that terms presented in this section are defined differently by a dictionary or other sections hereof, then the definition presented in this section shall govern in interpreting this specification and the accompanying claims.

“Ab” is an abbreviation for antibody.

The term “anti-Coccidioides antibody” as used herein include antibodies in the form of an intact IgG (IgG) or fragments of an IgG including F(ab′)₂ fragments and F(ab) fragments.

“BSA” is an acronym that stands for bovine serum albumin.

“Coefficient of variation” is an attribute of a distribution, i.e., the standard deviation of the distribution divided by its mean, and is typically expressed as a percentage. Coccidioides spp and Coccidioides are used interchangeably herein.

“CSF” is an acronym that stands for cerebrospinal fluid, which is a physiological specimen that can be tested for presence of Coccidioides antigen using the present invention.

“ELISA” is an acronym that stands for enzyme-linked immunosorbant assay; also abbreviated as “EIA,” which is used interchangeably with ELISA.

“F_{c}” means a highly conserved, non-antigen-binding fragment of an immunoglobulin obtained following papain digestion of an immunoglobulin.

“F(ab′)₂” means a bivalent antigen-binding fragment obtained following pepsin digestion of an immunoglobulin.

“F(ab)” means a monovalent antigen-binding fragment obtained following subjecting a F(ab′)₂ to a reducing agent.

“GARA” is an acronym that stands for goat anti-rabbit antibodies.

“HARA” is an acronym that stands for human anti-rabbit antibodies.

“HRP” is an acronym that stands for horseradish peroxidase.

“IgG” is an acronym that stands for immunoglobulin G, which is a class of antibodies found in serum.

“NRS” is an acronym that stands for normal rabbit serum, meaning serum from a non-immunized rabbit, particularly with reference to an antigen derived from Coccidioides.

“OD” is an acronym that stands for optical density, the subscript of which indicates a wavelength or wavelengths that are used to determine degree of color change, for example, caused by a reaction.

“PBS” is an acronym for phosphate-buffered saline

“TBS” is an acronym for Tris-buffered saline “RIA” is an abbreviation for radioimmunoassay.

“SD” is an acronym that stands for standard deviation, which is a measure of the variability of the distribution of data around the mean.

Spp means species as used herein

“UF” is an abbreviation for ultrafiltration, which is a filtering process that generally separates particles sized between 0.1 to 0.005 microns.

Hyphae and mycelia are used interchangeably herein.

### Enzyme-Linked Immunoassays

The present disclosure describes enzyme-linked immunoassay (“ELISA”), kits, procedures and diagnostic methods for identifying Coccidioides in an analyte. Also disclosed are methods of diagnosing a coccidioidomycosis.

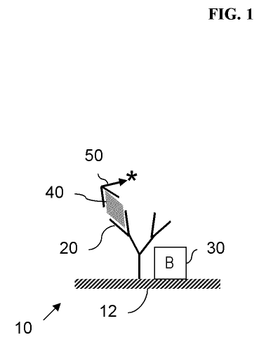

FIG. 1 is a graphical representation of a sandwich ELISA assay. As shown in FIG. 1, the capture antibody 20 is bound to the surface of a microtiter plate subsequently blocked with blocking reagent 30. The capture antibody is shown binding to an antigen 40 where the antigen is also bound to a detection antibody 50.

One embodiment according to the present disclosure is a method for the detection of a Coccidioides antigen wherein a binding surface is contacted with a capture antibody and then the binding surface is blocked with a blocking agent to yield an antigen binding surface. The antigen binding surface is then contacted with an analyte under conditions suitable to allow for the binding of a Coccidioides antigen to the antigen binding surface and then contacted with a detector antibody.

The binding surface can be any surface that is suitable for ELISA assays such as a plastic microtitre plate. The capture antibody is an antibody that is generated by vaccinating an animal with a Coccidioides antigen. In one embodiment according to the present disclosure the capture antibody is an unmodified polyclonal rabbit anti-Coccidioides IgG.

In one embodiment according to the disclosure the blocking agent is an animal serum other than BSA. In another embodiment the blocking agent may be a plant protein block agent or a blocker such as StartingBlock TBS which is marketed as being free of potentially interfering serum proteins (Thermo Fisher Scientific, Rockford, Ill.).

An embodiment according to the present disclosure is an ELISA assay in which there is both a capture antibody and a detector antibody that are directed to Coccidioides antigens. In one embodiment both the capture antibody and the detector antibodies are polyclonal rabbit anti-Coccidioides IgG. In another embodiment according to the present invention the capture antibody comprises an unmodified polyclonal rabbit anti-Coccidioides spp. IgG, and the detector antibody is a modified polyclonal rabbit anti-Coccidioides spp. IgG that does not comprise an F_{c} antibody portion, e.g. an F(ab′)₂ or F(ab) fragment of the antibody.

The capture and detection antibodies for ELISA assays according to the present disclosure are obtained by immunizing a suitable host animal, such as a rabbit, with a mixed vaccine comprising antigens obtained from multiple patient isolates. For example, capture and detection antibodies may be isolated from rabbits immunized with a vaccine comprising five Coccidioides mould isolates which were obtained from different patients over a 6-7 month period.

Preferably, capture and detection antibodies are obtained from rabbits after injection with a vaccine comprising two Coccidioides antigens, more preferably 2, or 3, or 4, or 5, or 6, or 7, or 8 Coccidioides. Polyclonal rabbit anti-Coccidioides antibodies according to the present disclosure may be generated against Coccidioides antigens including mould isolates or galactomannan purified from Coccidioides mould. As used herein, rabbit anti-Coccidioides antibodies may include antibodies isolated from rabbits immunized with either mould isolates or with galactomannan.

In an embodiment according to the present disclosure the capture antibody is isolated from one or more rabbits immunized with a vaccine comprising two or more Coccidioides mould isolates and the detector antibody is isolated from one or more rabbits immunized with a vaccine comprising galactomannan purified from two or more Coccidioides. In another embodiment the capture antibody is isolated from one or more rabbits immunized with a vaccine comprising, galactomannan purified from two or more Coccidioides and the detector antibody is isolated from one or more rabbits immunized with a vaccine comprising two or more Coccidioides mould isolates. In some embodiments of the present invention, the capture antibody is isolated from a rabbit that was immunized with a vaccine comprising 2, or 3, or 4, or 5, or 6, or 7, or 8, Coccidioides mould isolates. In some embodiments of the present invention, the capture antibody is isolated from a rabbit that was immunized with a vaccine comprising galactomannan purified from 2, or 3, or 4, or 5, or 6, or 7, or 8 Coccidioides mould isolates. In some embodiments of the present invention, the detector antibody is isolated from a rabbit that was immunized with a vaccine comprising 2, or 3, or 4, or 5, or 6, or 7, or 8, Coccidioides mould isolates. In some embodiments of the present invention, the detector antibody is isolated from a rabbit that was immunized with galactomannan purified from 2, or 3, or 4, or 5, or 6, or 7, or 8 Coccidioides mould isolates. In other embodiments, antibodies from two or more immunized rabbits may be combined for use in the assays according to the present disclosure.

Capture and/or detector antibodies are obtained from animals demonstrating at least 40%-50% or greater inhibition of the binding of a detector antibody detected in a test binding assay compared to the binding of the detector antibody in a control binding assay. Once isolated from the animal, the capture antibodies are attached to an immunoassay detection plate by any suitable method. Such attachment can be accomplished by the physical adsorption of the capture antibody to the surface, by action of van der Waals forces or hydrophobicity or the like. It is generally known among those skilled in the art that proteins generally, and certainly antibodies, have an affinity for plastic or glass surfaces, which are preferred surfaces used in the context of the present invention. Other preferred surfaces include polymers, both natural, such as cellulose or chitin and the like, and synthetic, such as nylon and the like. The most preferred surface used in the context of the present invention is a plastic surface. One could also attach the capture antibody to the surface by use of reactive groups that are themselves attached to the surface and that react covalently with the capture antibody.

The capture antibody is preferably an antibody derived from an animal, such as a rabbit, wherein the antibody binds to at least one Coccidioides antigen. A particularly preferred capture antibody is a rabbit-derived anti-Coccidioides IgG antibody. Optionally, the capture antibody can be modified by removing the F_{c} portion, as described in detail below with respect to the detector antibody to yield an F(ab) or F(ab′)₂ fragment of the antibody. The antigen binding surface can have any suitable concentration of the capture antibody.

Non-specific binding to an antigen binding surface is blocked by contacting the detector surface with a blocking reagent. In some embodiments, the blocking composition is substantially free of bovine serum albumin. In some embodiments the blocking agent may be a plant protein block agent or a blocker such as StartingBlock TBS which is marketed as being free of potentially interfering serum proteins (Thermo Fisher Scientific, Rockford, Ill.).

The antigen binding surface comprising a capture antibody is typically contacted with a blocking agent prior to contact with an analyte. The blocking agent is desirably provided as an excess of a suitable compound that will attach to the antigen binding surface in a manner that substantially reduces or prevents non-specific binding (i.e., binding of materials other than the analyte to the surface, either to the bound capture antibody or other binding sites). Preferably, the blocking agent does not itself attract specific or nonspecific interactions with the antigen of interest or antibody directed thereto.

After contacting the antigen binding surface with the blocking agent, an analyte is placed in contact with the antigen binding surface. The analyte typically comprises a physiological specimen containing an unknown amount of an antigen, a positive control known to contain the antigen, or a negative control known to not contain the antigen. The physiological specimen used in the context of the present invention is any specimen that may be collected from a patient. Preferably, the specimen is either a fluid when removed from the patient or tissue that has been macerated or soaked in a physiological saline buffer. Preferably, the physiological specimen is selected from the group consisting of serum, urine, cerebrospinal fluid, bronchioalveolar lavage fluid, pleural fluid, pericardial fluid, peritoneal fluid, synovial fluid, ocular fluid, and abscess contents.

In another embodiment, refinements in immunoassays pertain to detection antibody configurations permitting a reduction in false-positive or false-negative readings and/or an increase in the number of suitable sources of patient samples. Some patients may have developed anti-rabbit antibodies (HARA) before a sample, such as urine or blood, is taken from the patient. The presence of such antibodies can result in false-positive or false-negative results. In particular, detection antibodies are preferably combined with an animal serum that is selected based on a serum screening process to identify serum samples characterized by the ability to block or reduce interference by agents that are capable of causing false-positive or false-negative results. For example, rabbit serum can be screened to identify serum from rabbits that reduces interference by Goat Anti-Rabbit Antibody (GARA) (e.g., during serum screening described below) and HARA (e.g., during clinical testing) in ELISA detection.

Some embodiments according to the present disclosure are methods of detecting Coccidioides antigens in which the detection antibody is combined with an excess of animal serum to reduce false-positive results. In one embodiment the detection antibody is rabbit IgG or an IgG fragment, such as a F(ab′)₂ or F(ab) fragment and the animal serum is normal rabbit serum (NRS). In some embodiments the detector antibody is combined with NRS prior to contact with a capture antibody. Surprisingly, significant variation was observed in the ability of NRS obtained from different rabbits to reduce false-positive results. In certain embodiments the animal serum is selected by a screening method to select serum that reduces background reactivity and reactivity that causes false-positive or false-negative results false-positive false-negative. The animal serum is obtained from an unimmunized animal. For example, a screening assay to evaluate serum for use in the Coccidioides antigen assay might use an anti-rabbit antibody generated in a different species, for example goat (goat anti-rabbit antibody, GARA). The assay allows evaluation of the ability of that serum to block the interaction of GARA in sandwich immunoassays using the polyclonal anti-Coccidioides rabbit capture antibodies and a detector antibody. Variation in the ability of serum obtained from different rabbits to block false-positive results or false-negative results caused by GARA is evaluated in a series of immunoassays with a GARA control. The high and low Coccidioides antigen positive control samples are patient urine samples that have been adjusted to have a high or a low Coccidioides antigen level respectively. Ideally a rabbit serum sample selected for use as a blocker of undesirable interference exhibits maximal detection of Coccidioides antigen (a high optical density for the high and low positive Coccidioides antigen controls) and a low optical density in the GARA control. Results from an assay corresponding to the description above are shown in table 7 below.

Accordingly, an immunoassay preferably comprises the step of preparing a detector antibody composition comprising an animal serum screened for ability to reduce interference with detector antibody binding caused by GARA. In particular, the detector antibody is preferably combined with a serum that reduces the binding of GARA to a capture antibody but does not itself cause a false-positive or false-negative result. Preferably, the binding of the detector antibody to the bound antigen is not reduced by the presence of the serum. More preferably, the serum reduces the binding level of GARA to a capture antibody to less than 3.0-times, more preferably less than 1.5-times the binding level observed with the Coccidioidies negative control. Preferably the binding level of GARA to a capture antibody is no higher than observed with the Coccidioidies negative control. The serum is preferably derived from an unimmunized animal of the same animal species as the source of the detector antibody and/or the capture antibody. Normal rabbit serum is one particularly preferred serum. The immunoassay can therefore comprise the step of performing a serum screening assay to identify serum samples that desirably reduce false-positives, as indicated by the ability of the serum to increase detector antigen binding to a capture antibody in the presence of GARA. A serum screening assay preferably comprises one or more of the following steps: (a) providing a serum sample, (b) combining the serum with a detector antibody to form a detector antibody solution, (c) providing immunoassay test plates having a capture antibody attached thereto, (d) contacting the detector antibody solution with immunoassay test plates in the presence of a negative control, a Coccidioides antigen positive control or a control GARA positive control, (e) separately detecting the binding of the detector antibody to the immunoassay test plate in the negative control, the positive control and the GARA control, and (f) selecting the serum for inclusion in a detector antibody composition if binding of the detector antibody to the GARA control was inhibited in the presence of the serum sample. Preferably, the serum combined with the detector antibody does not inhibit the binding of the detector antibody to the Coccidioides positive control.

In another embodiment, the detection antibody is a modified IgG antibody that does not comprise the crystalline F_{c} domain for example an F(ab′)₂ or an F(ab) fragment of the IgG. An immunoglobulin structure consists of an antigen-binding domain (“F(ab′)₂”) and a highly conserved crystalline domain (“F_{c}”), which can be separated by proteolytic digestion with papain to obtain the F_{c} fragment or pepsin to obtain the F(ab′)₂ fragment. Alternatively, the intact IgG molecule may be used instead of a modified IgG antibody or fragment of an IgG antibody. The F_{c} fragment has a very similar amino acid sequence among all immunoglobulin G (“IgG”) molecules of at least the same species; in contrast, the F(ab′)₂ portion has both hypervariable as well as highly conserved regions when compared from antibody to antibody. The F(ab′)₂ portion comprises two F(ab) fragments attached to one another by disulfide bonds. Accordingly, a preferred detector antibody used in the context of the present invention is a F(ab) or a F(ab′)₂ fragment. Another preferred antibody derivative would retain the hypervariable regions found on the F(ab) structure but would have removed therefrom, or have masked, the constant regions found thereon. The capture or detector antibody can be a monoclonal, or a polyclonal, or a cloned nucleic acid that encodes the recognition site of the antibody of interest, from the same or a different species of animal than the capture antibody. Preferably, the antibodies used are monoclonal or a polyclonal antibodies; more preferably, the antibodies are polyclonal antibodies; most preferably, the antibodies are polyclonal IgG antibodies.

In general, the capture antibody, the detector antibody, and the animal serum can be derived from the same or different animals that have an immune system, which animals are individually of the same or different species. In preferred embodiments, the species of the animal in which the capture antibody is raised is preferably derived from a polyclonal preparation from rabbit origin. The detector antibody is preferably of rabbit origin as well. In embodiments where the detector antibody is administered in combination with a screened animal serum, the serum is typically derived from a non-immunized animal of the same species, preferably a rabbit. An alternative antibody source is of mouse origin, such as a monoclonal detector antibody directed to an epitope of a Coccidioides antigen where the antibody has suitable affinity for use in the immunoassays according to the present disclosure.

Detector antibodies and reporter molecule systems are well known in the art. One of skill in the art would be able to choose a system that is suitable for a particular purpose. Briefly, the detector antibody preferably recognizes the antigen of interest and is adapted to bind to a reporter molecule. Optionally, a portion of the detector antibody itself can be detected. Typically however, a portion of the detector molecule is capable of high-affinity binding to a reporter molecule that can be readily detected. For example, the detector antibody can be adapted to bind to a reporter molecule by linking the detector antibody to a biotin moiety, which is able to form a high affinity link with a reporter element comprising a streptavidin moiety. Other linking means for joining a reporter element to the detector antibody include, without limitation, sulfosuccinimidyl-4-N-maleimidomethyl-cyclohexane-1-carboxylate (Sulfo-SMCC), sulfosuccinimidyl-6-3′-2-pyridyldithio-propionamido-hexanoate (Sulfo-LC-SPDP), N-maleimidobutyrloxy-sulfo-succinimide ester (Sulfo-GMBS), and the like; two complementary segments of DNA; and a lectin and an appropriate sugar. Unbound detection antibodies can be removed by washing the surface with a wash solution, commonly a neutral saline solution. The wash step at this point in the ELISA protocol is optional depending on whether the protocol is a heterogeneous or homogeneous format.

After contacting a suitable detection enzyme composition with an antigen binding surface under conditions permitting the detection antibody to bind to the surface bound antigen, a composition comprising a reporter element molecule can be contacted with the bound detection antibody. The reporter element molecule is preferably adapted to bind to the detector antibody with a high affinity. For example, when a biotinylated detector antibody is used, a streptavidin-bound reporter element molecule such as horseradish peroxidase can be used. The reporter element composition is added under conditions to permit, or preferably to promote, binding of the detector antibody to the reporter element molecule to form a reporter-conjugated matched pair molecule. Subsequently, unbound reporter-elements can be removed by washing the surface with the same or similar wash solution. This wash step is particularly preferred for either hetero- or homogeneous ELISA formats, as the enzyme conjugated to the matched pair (e.g., the biotin-streptavidin combination) is the signal generator by which the ELISA test is assessed, as further described below.

Preferably, the reporter element-conjugated matched pair component includes an enzyme or a tag that generates a signal by itself (in the case of a fluorescent or radioactive tag) or in the presence of a substrate (in the case of certain enzymes), which signal is commonly a pigment, or visible light, or fluorescence, or radioactivity. Preferred enzymes used in the context of the present invention include, without limitation, a peroxidase, alkaline phosphatase, beta-galactosidase, chloramphenicol acetyl transferase, and/or a luciferase (e.g., that of renilla or a firefly). Preferred substrates for such enzymes include, without limitation, luciferin, tetramethylbenzidine, diethanolamine, p-nitrophenol phosphate (PNPP), 2,2′-azino-bis[ethylbenzthiazoline-6-sulfonic acid] (ABTS), o-phenylenediamine dihydrochloride (OPD), 2-Nitrophenyl-b-D-galactopyranoside (ONPG), 4-Nitrophenyl-b-D-glucuronide (NPG). Preferred dyes, fluorescent tags, metal tags, radioactive tags, and the like, include: fluoroscein, rhodamine, Texas Red, Cy dyes, R-phycoerythrin, gold, PBXL, magnetic microparticle, and latex microparticle, each of which can be covalently linked to a component of either component of the matched pairs. The method preferably further includes detecting a signal, which includes any or all of detecting or measuring light or radioactive emission, dye generation, color change, magnetic or metallic bound components, light scattering, and the like.

Another embodiment according to the present disclosure is an immunoassay method and kit for detecting a Coccidioides antigen. The method permits detection of Coccidioides antigen in a patient sample comprising HARA with a low incidence of false-positive or false-negative results. The kit comprises an ELISA microtiter plate comprising an antigen binding surface, and a detection antibody composition. Most preferably, a kit comprises a screened animal serum and a detector antibody comprising a modified IgG antibody. The modified IgG detector antibody may be a F(ab′)₂ fragment or a F(ab) fragment. The detector antibody is preferably adapted to couple with a reporting element, for example by a biotin-streptavidin linkage. The kit may further comprise control samples for high and low positive readings, as well as a negative sample. Control samples may be obtained from clinical specimens or other sources. A blocking agent having a desirably low coefficient of variation can also be included, such as a blocking agent substantially free of BSA. A set of suitable reporting reagents, such as HRP, TMB and H₂SO₄, may also be included to provide a means for detecting bound detector antibody.

To form the microtiter plate, a capture antibody is obtained from a suitable animal, such as a rabbit, obtained after vaccinating the animal with two or more, preferably 2-5, different strains of Coccidioides antigen in any suitable manner. The capture antibody is preferably an unmodified anti-Coccidioides IgG rabbit antibody, but may also be a modified IgG antibody having the F_{c} portion truncated or removed. The capture antibody can be immobilized on an ELISA microtiter plate or any suitable platform by any suitable method to form a detection surface. Next, the detection surface is contacted with a suitable blocking agent. The blocking agent may be substantially free of BSA. In some embodiments the blocking agent may be a plant protein block agent or a blocker such as StartingBlock TBS which is marketed as being free of potentially interfering serum proteins (Thermo Fisher Scientific, Rockford, Ill.). Samples containing positive controls, negative controls or samples for analysis can be contacted with the antigen binding surface in the microtiter ELISA plate in any suitable manner permitting antigen binding to the capture antibodies. Optionally, unbound antigen can be rinsed from the antigen binding surface. Subsequently, the detector antibody composition can be added to the microtiter plate in a manner permitting the detector antibody to bind to surface bound antigen. Unbound detector antibody can be removed from the antigen binding surface and a reporter element can be added to the microtiter plate to attach the reporter element to detector antibodies attached to bound antigen. The reporter element can be stabilized in the blocking composition, preferably comprising non-serum proteins. Finally, the reporter element can be detected using any suitable method, including radiodetection or optical density detection.

### Quantitative Immunoassay Reporting

In another embodiment, a method of quantitative reporting of immunoassay results is provided. Typically, the detection of antigen levels in immunoassays are expressed semi-quantitatively by comparison of the amount of bound detector antibody in the presence of an analyte with a cutoff for positivity, determined by comparison to a negative or a positive control. Accordingly, the amount of antigen in a sample is typically reported in antigen units (for example, by dividing the detected antigen signal by the cutoff or the negative control). However, due to day-to-day variability in antigen assay measurements, it is usually necessary to test a prior sample along with the current sample in the same assay to assess the change in antigen during treatment. In contrast, methods for determining antigen concentration by comparison to a calibration curve are provided herein, wherein antigen concentration is provided in units of concentration (e.g., ng/mL) rather than antigen units. Dilutions of a urine pool from patients with coccidioidomycosis, determined to contain known amounts of Coccidioides galactomannan, by comparison to purified Coccidioides mycelial galactomannan, is preferably selected as a calibration standard for quantitative reporting of antigen detection.

### Example 1

This example illustrates an ELISA diagnostic test directed at Coccidioides antigen. Experimental physiological specimens and storage thereof.

Records were reviewed from 10 patients with coccidioidomycosis who had specimens for Histoplasma antigen testing performed at MiraVista Diagnostics, Indianapolis, (Kuberski et al. Clin. Infect Dis 2007; 44:e50-e54). The patients were from three clinical practices, two in Phoenix, Ariz. and one in Los Angeles, Calif. The authors were the treating physicians in all cases. Coccidioidomycosis was classified as acute if symptoms were present for less than 14 days and chronic if symptoms had been present for more than 14 days. The specimens obtained in 2005 and 2006 and were stored frozen and −20° C. Control physiological specimens for testing the standard ELISA. Control physiological specimens used to test the ELISA were urine specimens from the following individuals:

10 healthy laboratory personnel;

8 patients with histoplasmosis

The ELISA method. Unless otherwise indicated, reagents and chemicals for making buffers and solutions of antibodies or enzymes or substrates were purchased from Thermo Scientific, Rockford, Ill. The wells of Immulon-2 microtiter plates (rigid plates whose wells have flat bottoms;) were coated with 100 μl of an immunoglobulin G (IgG) fraction of rabbit anti-Coccidioides serum (the capture antibody) in 0.01 M Tris-HCl (pH 7.0) (0.01 M Tris), incubated at 37° C. for 1 hour, and washed with phosphate-buffered saline (pH 7.2) containing 0.05% polyoxyethylene (20) sorbitan monolaurate (sold under the tradename “Tween® 20” by EMD Biosciences Inc., San Diego, Calif.). The wash buffer was made fresh daily. Two hundred microliters of a plate blocking buffer (i.e., 5% bovine serum albumin in 0.01 M Tris) was added to each well, and the plate was incubated and washed as described above.

Next, 100 μl of undiluted urine was added to each well, and the plate was incubated and washed as described above. The wells were incubated with 100 μl of rabbit anti-Coccidioides IgG conjugated to biotin (the detector antibody) in 0.1 M Tris-HCl (pH 8.0) (0.1 M Tris) and washed as described above.

Finally, Coccidioides antigen adhering to the solid-phase antibody was measured by adding 100 μl of streptavidin-horseradish peroxidase in 0.1 M Tris-5% bovine serum albumin to each well. The plate was incubated and washed as described above. 100 uL Peroxidase substrate (TMB 1, BioFX, Owings Mills, Md.; was added to each well. Color development was stopped by the addition of 100 μl of 1.0 M H₂SO₄ to each well, and the optical density of the plate was read on a microplate-reading spectrophotometer (e.g., Sunrise Microplate reader, Tecan, Durham N.C.) at a dual wavelength of 450 nm/620 (“OD₄₅₀”). Results that were two times higher (cut off for positivity) than the mean value for normal, negative samples were considered positive. All results were divided by 2 times the mean value for the normal urine samples and were expressed as EIA units. They were reported as ng/mL based upon extrapolation from a calibration curve.

The Histoplasma antigen EIA method. The specimens also were tested in the Histoplasma antigen EIA, as previously reported (Kuberski et al. Clin. Infect Dis 2007; 44:e50-e54).

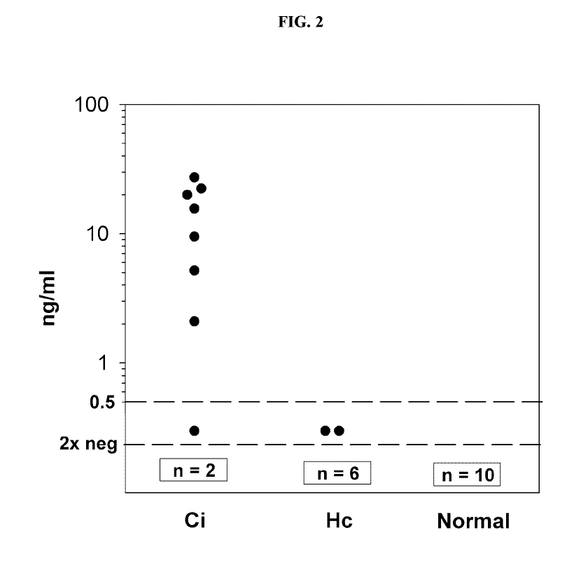

Sensitivity and specificity. A total of 10 urine specimens from patients with coccidioidomycosis and 18 control specimens were tested. Coccidioides antigen was detected in 8 of the 10 (80%) coccidioidomycosis patients compared to 2 of 8 (25%) histoplasmosis cases, but none of 10 controls. The 2 histoplasmosis patients that were positive in the Coccidioides antigen EIA exhibited the highest levels of Histoplasma antigen, >39 ng/ml and 34.78 ng/ml. Results are shown in FIG. 2.

Only 5 of the 10 coccidioidomycosis specimens were positive in the Histoplasma antigen EIA, demonstrating the improved sensitivity for detection of Coccidioides antigen using the Coccidioides antigen EIA.

Reproducibility. The reproducibility of the ELISA method was examined by testing specimens from 5 coccidioidomycosis patients and controls in 5 wells each on 5 consecutive assays within a period of one week. As shown in Table 1, the intraassay CV ranged 1.7% and 17.4% from and interassay between 6.4% and 12.7%. Intraassay and interassay reproducibility

TABLE 1

| | Intraassay | | | | | Interassay | | | | |
|---|---|---|---|---|---|---|---|---|---|---|
| Parameter | 1¹ | 2 | 3 | 4 | 5 | 1 | 2 | 3 | 4 | 5 |
| Mean-ng/mL | 8.04 | 2.70 | 0.63 | 0.13 | 0.44 | 7.97 | 2.62 | 0.67 | 0.13 | 0.48 |
| SD²- | 1.40 | 0.12 | 0.04 | 0.002 | 0.08 | 1.01 | 0.19 | 0.04 | 0.012 | 0.06 |
| ng/mL | | | | | | | | | | |
| CV³-% | 17.4 | 4.6 | 6.3 | 1.7 | 17.3 | 12.7 | 7.3 | 6.4 | 9.5 | 12.3 |

¹Patient number;
²standard deviation; coefficient of variation,
³expressed as percentage.

Conclusion. Accordingly, the sensitivity for detection of Coccidioides antigen is 80%, and appears to be higher than in the Histoplasma antigen EIA (50%). Cross reactions occurred in only 25% of histoplasmosis cases, compared to 100% in the Histoplasma antigen EIA. The assay is promising for diagnosis of coccidioidomycosis.

### Example 2

To evaluate the desirability for using F(ab′)₂ as the detector antibody, the following experiment was performed. The IgG and F(ab′)₂ were evaluated with the high and low positive Coccidioides antigen controls, as well as a GARA control. As an additional measure to reduce interference caused by anti-rabbit antibodies, the biotinylated IgG and F(ab′)₂ were studied in the presence or absence of NRS. In all other respects, the procedure set forth in Example 1 was employed. The data are presented Table 2.

TABLE 2

| Detector | | | | | |
|---|---|---|---|---|---|
| Antibody | Neg | Hi Pos | Low Pos | GARA | Cut Off |
| B-IgG | 0.036 | 1.742 | 0.384 | 1.559 | 0.072 |
| B-IgG + | 0.032 | 1.807 | 0.403 | 0.042 | 0.064 |
| NRS | | | | | |
| B-F (ab′)₂ | 0.143 | 1.275 | 0.300 | 0.917 | 0.286 |
| B-F (ab′)₂ + | 0.042 | 1.288 | 0.245 | 0.046 | 0.084 |
| NRS | | | | | |

Biotinylated IgG (B-IgG) in the presence of NRS provided a high sensitivity for detection of Coccidioides antigen and the lowest reactivity with the GARA control. The background activity in the negative control was significantly higher using Biotinylated F(ab′)₂ (B-F(ab′)₂) without NRS. Accordingly, the best results were obtained by using biotinylated IgG diluted in NRS as the detector antibody.

### Reagents.

(1) Blocking agents. StartingBlock TBS (Catalog #37542, Thermo Fisher Scientific, Rockford, Ill.) and StartingBlock PBS (Catalog #37538, Thermo Scientific, Rockford, Ill.) were purchased, both of which are protein-based in either a tris-buffered or phosphate-buffered saline. PBS blocking solution is used undiluted from the bottle to coat the plates; coated plates are not used after 90 days; TBS assay diluent is used undiluted from the bottle, and stored at 4° C.

(2) Biotin-conjugated anti-Coccidioides antigen rabbit F(ab)₂ and IgG. A F(ab′)₂ fragment was created by pepsin digestion of the Protein-A purified IgG rabbit polyclonal antibody, which was then conjugated to biotin (i.e., F(ab′)₂ to biotin). A working dilution of the F(ab′)₂ is made by diluting in StartingBlock TBS, to a final concentration of 200 to 300 ng/ml as determined by titration. In this way, we optimized discrimination of positive and negative controls, and maintained the negative control optical density at 450/630 nm below 0.10.

(3) Normal rabbit serum (NRS). Serum from Flemish Giant rabbits was collected twice monthly and pooled to form NRS used in the diluent for the detector antibody. NRS is aliquoted and stored at −80° C. Prior to use, the NRS is combined with the diluted biotinylated F(ab′)₂ or IgG to a final concentration of 5%.

(4) Streptavidin-HRP conjugate. Streptavidin-HRP is purchased lyophilized from Roche (Catalog #1089153, Streptavidin-POD); reconstituted in 1.0 ml of autoclaved ultrafiltration (UF) filtered water. Reconstituted streptavidin-HRP is replaced after no more than 6 months and is stored at 4° C. A working dilution is made by diluting in StartingBlock TBS, 1:50,000 from the stock.

(5) Color generation system. TMB Microwell Peroxidase Substrate System (BioFX TMB1) was purchased from KPL (Gaithersburg, Md.). This is a single component system and must be at room temperature prior to use. The chromogenic substrate needs to be protected from light to avoid degradation. Substrate system is stored refrigerated at 4° C.

(6) 0.01 M Tris-HCl Buffer. To prepare a 10× stock, combine: (i) 12.1 g Tris Base (Sigma Chemicals, St. Louis; Catalog #T-8524); (ii) add 900 ml UF filtered water to dissolve; (iii) pH to 7.0 with concentrated HCl; and (iv) bring final volume to 1000 ml with UF filtered water. Store at 4° C. Stock is replaced after no more than 6 months. Before use, dilute to 1× with UF filtered water. The buffer is stored refrigerated at 4° C.

(7) 0.1 M Tris-Saline Buffer. To prepare 10× stock, combine: (i) 12.1 g Tris Base (Sigma, Catalog #T-8524); (ii) 85.0 g NaCl (Sigma, Catalog #F-9625); (iii) add 900 ml UF filtered water to dissolve; (iv) pH to 8.0 with concentrated HCl; and (v) bring final volume to 1000 ml with UF filtered water. Store at 4° C. Stock is replaced after no longer than 6 months. Before use, dilute to 1× with UF filtered water. The buffer is stored refrigerated at 4° C.

(8) PBS EIA Wash. PBS with Tween® 20 tablets (EMD Biosciences), are prepared according to package instructions. One tablet dissolved in 1 liter of UF flittered water prepares 10 mM phosphate butter, pH 7.4, 140 mM NaCl, 2.7 mM KCl, 0.05% Tween 20. The wash solution is made fresh daily.

(9) 2.0 N Sulfuric Acid (LabChem. Inc., Catalog #LC25790-1).

(10) Positive Controls. The positive controls consist of a High and a Low Positive of concentrated known Coccidioides antigen positive urine. High Positive Control Urine is diluted 1:1 from stock−50 ml concentrated urine+50 ml 0.1 M Tris. Low Positive Control Urine is diluted 1:32, i.e., 3.125 ml High Positive Control urine+96.875 ml 0.1 M Tris. For quality control testing, new positive controls are tested in tandem with existing controls. New lots have at most 20% mean difference from the existing controls to be accepted. Aliquots currently in use are stored in the refrigerator. Aliquots of accepted controls not in use are stored frozen.

(11) Negative Controls and Cut-Off Calibrators. 0.1 M Tris-saline pH 8.0 serves as a blank. Calibrators were chosen such that the lowest calibrator is the zero calibrator, with the 0.07 ng/nL calibrator serving as the lower limit of quantification (LLQ).

ELISA procedure. Remove appropriate number of precoated plates from the sealed bags for the number of specimens to be tested. All wells not being used should be removed from the plate and returned with a desiccant to storage.

Add 100 μl/well of the control or specimen to be tested. All samples are tested in the following order on each plate: Cutoff calibrator, high positive control, low positive control, negative control, patient samples. Patient samples are loaded in order according to the work list.

Seal each plate and incubate at 37° C. for 1 hour. Then wash the plate five times with freshly prepared PBS EIA wash. Then add 100 μl/well of biotin-conjugated anti-Coccidioides rabbit B-F(ab′)₂ with 5% NRS in StartingBlock TBS. Reseal each plate and incubate at 37° C. for 1 hour.

Wash the plate five times with PBS EIA wash as above. Add 100 μl/well of a 1:50,000 dilution of HRP-labeled streptavidin in StartingBlock TBS. Reseal each plate and incubate at 37° C. for 1 hour. Wash the plate five times with PBS EIA wash as described above.

Add 100 μl/well of room temperature TMB1 Peroxidase Substrate (BioFX, Owings Mills, Md.). Develop the plate for 8 minutes at room temperature. Stop the reaction by adding 100 μl/well of 2.0 N sulfuric acid. Color development is measured by reading the optical density on the Tecan EIA Plate reader at a dual wavelength of 450/620 nm.

### Example 3

### Preparing Coccidioides Mould Isolates for Vaccine Preparation

A multiple-isolate rabbit vaccine was made using five Coccidioides mould isolated from urines samples obtained from coccidiodomycosis patients from California (C. immitis) and Arizona (C. posidasii).

The isolates were grown out of the urine samples by streaking 0.1 ml of urine on Yeast Extract Phosphate Agar w/Ammonia. Mould growth was eamined for the development of arthroconidia. Selected isolates were then grown on potato dextrose slants. All samples were confirmed as Coccidiodes.
1. In order to grow enough of each isolate to make a vaccine. Individual colonies were chosen from the Yeast Extract Phosphate Agar plate and were subcultured onto 2-3 Potato Dextrose Agar slants. When sufficient mould growth occurred, in approximately 2 weeks, flasks of Histoplasma macrophage media (HMM) broth were inoculated. Inoculate 12 sterile one liter flasks containing 400 ml sterile HMM with a pea sized volume of mould from a slant.
2. Place all 12 flasks into shaker incubator at 37° C., shaking at 150 rpm and allow to grow for at least 1 week.
3. Check for contamination of flasks by placing one drop on a slide (under the hood) with LPCB (lactophenol cotton blue) and examine under the microscope for a pure culture (lack of other moulds or bacterial contamination).
4. Filter sterilize the mould mat through a 0.22 um filter, then wash the mould mat with sterile UF filtered water until clean of culture supernatant.
5. When all the mould mats are washed in water, combine mould mats into 8 conical tubes.
6. Bring volume up to about 45 ml with PBS containing 5% Formalin. Place the conical tubes on the rotator overnight (18 hrs) at 4° C.
7. The next day, wash the mould suspensions with sterile saline or PBS to remove all the formalin. To ensure that all the formalin has been removed, wash at least 6 times or until a formalin test strip reads less than 2.5 PPM. Suspensions can be combined into 4 conical tubes at this point.
8. After the formalin has been removed, place the mould in a Waring blender and dilute to a 30% suspension with sterile saline. Blend thoroughly and aliquot into 20 ml aliquots in 50 ml tubes.
9. Add a few drops of the final suspension from each conical to either a Potato Dextrose Agar slant or plate to check for viability. After about 2 weeks the slants were checked for growth. No growth indicated that the mould was killed by the formalin.

### Example 4

### Preparation of Purified Coccidioides Galactomannan

Six clinical patient isolates were grown in 12×1.0 L flasks containing 400.0 ml of brain heart infusion broth incubated at 37° C. on a gyratory shaker (New Brunswick Scientific Co., Inc., New Brunswick, N.J.) for 72 h. The culture supernatant was separated from the mycelia by filtering through a sterile 0.2 um filter unit. Protease inhibitors and 0.02% sodium azide were added and the culture supernatant was stored at 4° C. until it was concentrated. Culture supernatant was concentrated 50×, then buffer-exchanged into UF filtered water in a KrosFlo Hollow Fiber Tangential Flow Module (Spectrum Laboratories, Rancho Dominguez, Calif.).

Galactomannan was precipitated from the concentrated supernatant by adding 4 parts 100% ethanol to 1 part concentrated supernatant. Ethanol/galactomannan solution was placed at −80° C. overnight then centrifuged at 4000×g for 20 minutes. The supernatant was removed and precipitated galactomannan was dissolved in UF filtered water. Galactomannan was purified by immunoaffinity chromatography using Con A Sepharose.

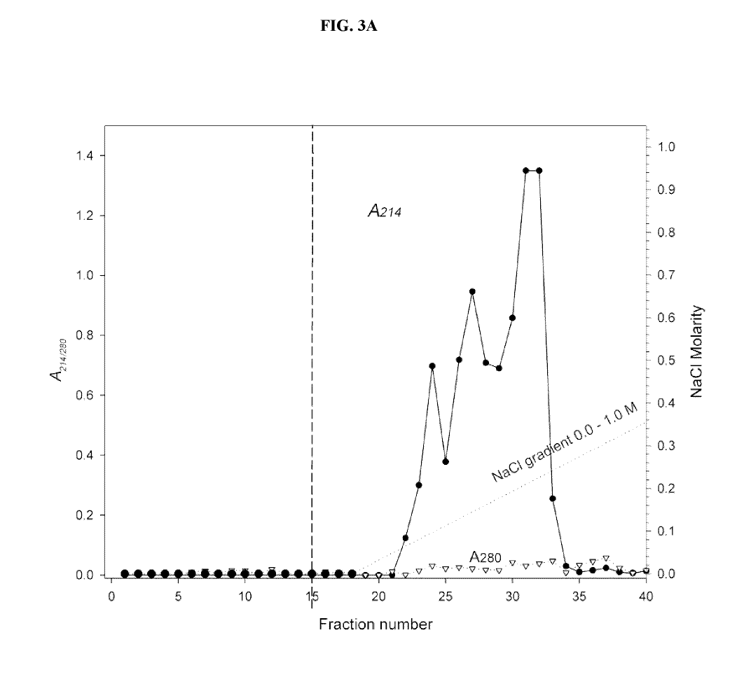

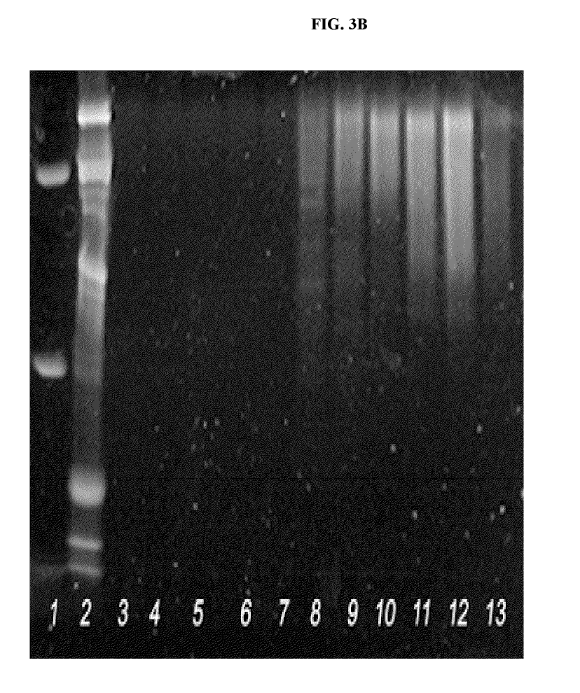

A 2.5×40.0 cm column containing 105.0 ml bed volume of Con A Sepharose was prepared according to the manufacturer's instructions (GE lifesciences, Piscataway, N.J.). The ethanol precipitated galactomannan was reconstituted in UF filtered water, filtered and diluted 1:1 in Con A buffer (0.2 M tris, 0.5 M NaCl, 1.0 mM MnCl₂, 1.0 mM CaCl₂) and loaded on the column. The loaded extract was allowed to react for 1.0 h and afterwards the column was washed with Con A buffer at a flow rate of 2.0 ml/min as controlled with an Aktaprime Plus Purification System (GE Healthcare, Piscataway, N.J.) The column was washed until the OD₂₁₄ values were back to baseline values, then with an additional 2 column volumes. The column was eluted using 0.3 M α-methyl-D-mannopyranoside in Con A buffer. 4 mL Fractions were collected and monitored at A₂₁₄, and in the previously described immunoassay. Based on these results, fractions exhibiting absorbance at A₂₁₄ and strong reactivity in the immunoassay were considered to contain a significant amount of antigenic material and were pooled. The pooled fractions were concentrated to a volume of <50.0 ml, using a KrosFlo Hollow Fiber Tangential Flow Module (Spectrum Laboratories, Rancho Dominguez, Calif.), galactomannan was then lyophilized and stored desiccated. FIG. 3A shows fractions from the DEAE column containing Coccidioides galactomannan. The galactomannan was contained in fractions 21-34. FIG. 3B shows an SDS PAGE profile corresponding to the data in FIG. 3A. The first lane contains lane markers and the other lanes contain DEAE fractions. Lanes 8-12 contains fractions 24-32, showing close correlation with the OD₂₁₄ carbohydrate peak.

### Example 5

### Preparation of Coccidioides Galactomannan Reference Calibration Standards

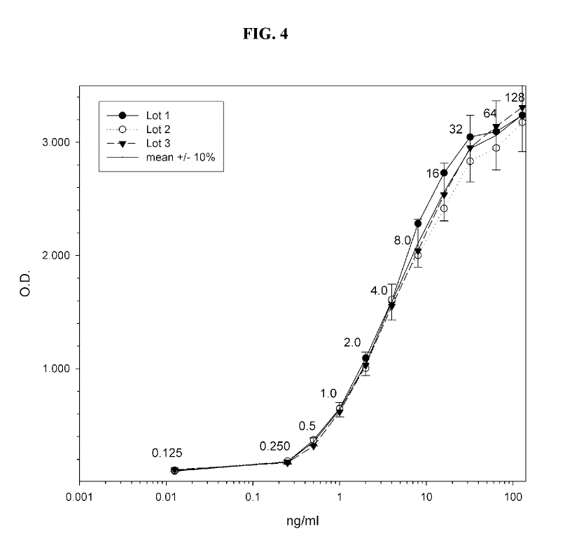

Reference calibration standards were prepared from the purified Coccidioides galactomannan antigen. Three lots of each of the calibrators were prepared by dilution of individually weighed amounts of the purified galactomannan and Starting Block, at concentrations of 128, 64, 32, 16, 8, 4, 2, 1, 0.5, 0.25, 0.125 ng/ml. The three reference standards were tested in the Coccidioides antigen EIA. As shown in FIG. 4, results were highly reproducible for the three reference standards.

### Example 6

### Determination of Antigen Concentration

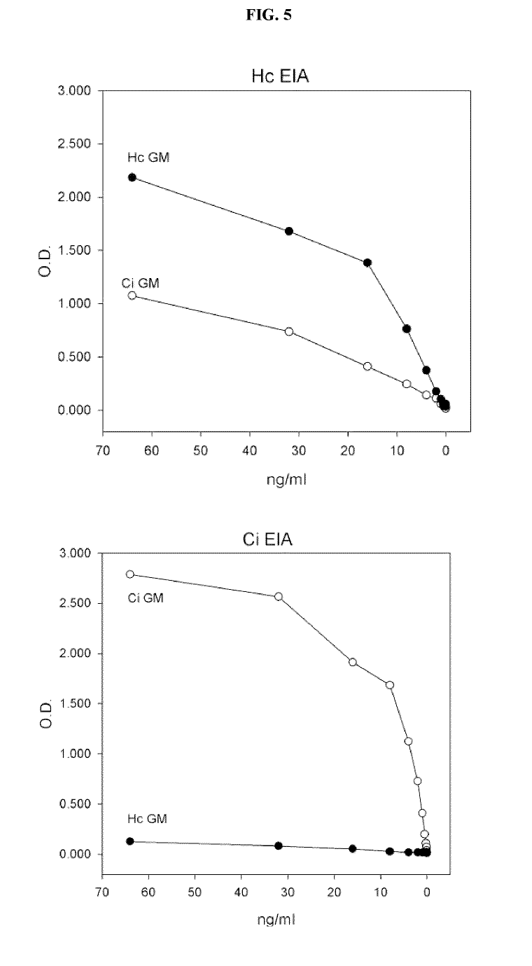

FIG. 5 shows a standard curve generated using calibration standards prepared from the urine from patients with coccidioidomycosis. The calibration curve is based on dilutions of a calibration standard comprising known amounts of a galactomannan, showing detection of purified galactomannan in a MVista® Antigen EIA.

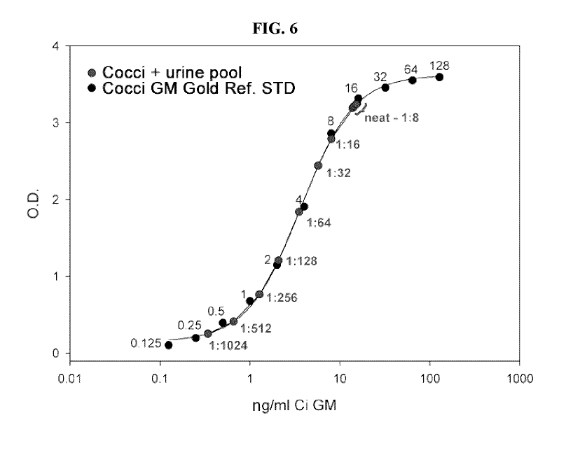

As shown in FIG. 6, the shape of the curves for the positive urine pool and the gold standard were superimposable, validating use of the positive urine pool calibrators for quantitation of antigen in clinical specimens. Accordingly, the urine calibrators were assigned ng/ml concentration values of 8.2, 5.9, 4.0, 1.9, 0.63, 0.31, 0.18, 0.07 and 0 ng/ml.

Results were classified as positive or negative by comparison of the optical density of the test specimen to that of the known antigen control specimen. Galactomannan concentration of specimens positive by comparison to the negative control was determined by comparing the optical density of the test specimen to that of the calibration curve standards, and results were expressed ng/ml.

### Comparison of Detection Levels of Coccidioides Galactomannan and Histoplasma Antigen in Both the Coccidioides EIA and the Histoplasma EIA

The lower limit of detection for the Coccidioides galactomannan was 0.03 ng/mL in the Coccidioides EIA versus 0.5 ng/mL in the Histoplasma antigen assay. The lower limit of detection for the Histoplasma galactomannan was 0.125 ng/ml in the Histoplasma EIA versus 8 ng/ml in the Coccidioides EIA. Thus, low concentrations of Coccidioides galactomannan (0.5 ng/mL) could be detected in the Histoplasma antigen EIA, explaining the high rate of cross-reactivity (59%) in patients with coccidioidomycosis. Conversely, high concentrations of Histoplasma galactomannan (8 ng/mL) were required for cross-reactivity in the Coccidioides antigen EIA.

### Example 7

Three sets of calibrations standards were prepared from a pool of urine specimens containing high levels of Coccidioides antigen. Urine specimens were first screened for cross-reactivity in the Platelia® Aspergillus galactomannan antigenemia assay (BioRad), and those that were positive were excluded from the calibrator pool. Multiple dilutions of the urine calibrator pool, which is designated as Cocci+urine pool in the figure, were prepared, and the antigen content of each was determined by comparison to known concentrations of the purified galactomannan (128, 64, 32, 16, 8, 4, 2, 1, 0.5, 0.25, 0.125 ng/ml), which is designated as Cocci GM Gold Ref STD.

Selection of Antibodies

Preferably, the capture and/or detector antibodies are obtained from animals demonstrating about a 40%-50% or greater inhibition in a test binding assay compared to the binding of the detector antibody in a control binding assay. The test binding assay comprises following steps: providing an antigen binding surface comprising anti-Coccidioides rabbit IgG capture antibody, contacting the antigen binding surface with a mixture of Coccidioides antigen and serum obtained from a vaccinated animal (preferably, a rabbit) in a manner effective to bind the Coccidioides antigen to the capture antibody, contacting the bound Coccidioides antigen with a detector antibody comprising a biotin moiety of the anti-Coccidioides rabbit IgG antibody, and detecting the bound detector antibody. The control binding assay is the same as the test binding assay, except that the serum is obtained from a non-vaccinated rabbit. The percent-inhibition is defined as the amount of bound detector antibody detected in the test binding assay divided by the amount of bound detector antibody detected in the control binding assay (e.g., (ODₜₑₛₜ/OD_{control})×100). Five of nine rabbits vaccinated with galactomannan and eight of nine vaccinated with mould antigen showed a percentage inhibition of 40% or greater, Table 3. Accordingly, detector and captive antibodies are preferably obtained from an animal vaccinated with two or more antigen samples, preferably from two or more Coccidioides antigens obtained from patient samples.

### Example 8

### Immunization of Rabbits and Selection of Antibodies for Use as Capture or Detector Antibodies Immunization of Rabbits with Galactomannan

Con A purified galactomannan described above was diluted in sterile PBS to a concentration of 360 ug/ml. Rabbit vaccine was prepared by emulsifying Freund's adjuvant) 50:50 with galactomannan. Rabbits were vaccinated monthly with a total of 1 ml of vaccine, and bled 7-10 days later. Freund's complete adjuvant was used for the primary immunization and incomplete adjuvant for the booster immunizations.

Immunization of Rabbits with Mould

Coccidioides mould described above in was emulsified 50:50 with Freund's adjuvant, as described above. Rabbits were vaccinated monthly with a total of 3 ml of vaccine, and bled 7-10 days later.

### Example 9

### Capture Assay to Evaluate Rabbit IgG for Detection of Coccidioides Antigen

Rabbit serum obtained from rabbits vaccinated with the multiple-isolate vaccine was evaluated in a capture binding assay according to the following steps:

A small amount of test IgG was purified from serum from each rabbit being evaluated using Immunopure A Plus IgG purification kit (Pierce, Rockford, Ill.) according to manufacturer's kit instructions. The concentration of each IgG was determined by a reading on the spectrophotometer at OD₂₈₀. Each test IgG along with positive control IgG was coated on microtiter plates at 25 ug/ml, according to the standard clinical assay plate preparation protocol. Specimens from two patients with coccidioidomycosis, one with histoplasmosis, and one normal subject were used to evaluate each new rabbit IgG. Results are provided in Table 3 below. Three patterns of reactivity are noted: high reactivity with Coccidioides antigen, high reactivity with Coccidioides antigen but cross-reactivity with Histoplasma antigen, poor reactivity with either antigen. These findings indicate that results of competitive assay identify antibodies that work well for detection of antigen in patient specimens. Based upon these findings, antibodies from rabbits 5052, 5053, 5057, 5058 and 5061 were selected for further analysis.

TABLE 3

| | GM vaccine | | | | | | | | |
|---|---|---|---|---|---|---|---|---|---|
| | 5051 | 5052 | 5053 | 5054 | 5055 | 5056 | 5057 | 5058 | 5059 |
| Inhibition-% | 1 | 68 | 59 | 43 | 5 | 10 | 50 | 66 | 33 |
| Cocci | 0.084 | 0.811 | 0.853 | 0.211 | 0.027 | 0.039 | 0.421 | 0.662 | 0.337 |
| patient 1 | | | | | | | | | |
| Cocci | 0.085 | 1.315 | 1.468 | 0.549 | 0.026 | 0.028 | 1.017 | 1.210 | 0.703 |
| patient 2 | | | | | | | | | |
| Histo patient | 0.064 | 0.102 | 0.033 | 0.015 | 0.013 | 0.013 | 0.028 | 0.014 | 0.027 |
| Normal | 0.059 | 0.056 | 0.02 | 0.01 | 0.007 | 0.005 | 0.01 | 0.008 | 0.019 |
| subject | | | | | | | | | |
| | Mold vaccine | | | | | | | | |
| | 5060 | 5061 | 5062 | 5063 | 5065 | 5067 | 5068 | 5069 | 3271 |
| Inhibition-% | 67 | 88 | 83 | 80 | 62 | 50 | 40 | 20 | 52 |
| Cocci | 0.876 | 1.416 | 2.006 | 2.218 | 1.268 | 1.142 | 1.461 | 0.283 | 1.972 |
| patient 1 | | | | | | | | | |
| Cocci patient 2 | 1.674 | 2.283 | 2.769 | 2.979 | 2.096 | 2.128 | 2.470 | 0.802 | 2.888 |
| Histo patient | 0.097 | 0.088 | 0.473 | 0.717 | 0.431 | 0.683 | 0.754 | 0.029 | 0.563 |
| Normal | 0.028 | 0.024 | 0.061 | 0.209 | 0.058 | 0.040 | 0.138 | 0.011 | 0.093 |
| subject | | | | | | | | | |

### Example 10

### Assessment of Antibodies from Rabbits Immunized with Coccidioides Mould or Galactomannan

As shown in Table 4, Coccidioides antigen was detected best in a hybrid assay using antibodies from rabbits immunized with Coccidioides mould for capture and galactomannan for detection.

TABLE 4
Coccidioides EIA using different capture or detector antibodies

| | Capture | | | | |
|---|---|---|---|---|---|
| | A (GM) | B (Mould) | C (Mould) | A (GM) | B (Mould) |
| | Detector | | | | |
| | A (GM) | A (GM) | A (GM) | B (Mould) | B (Mould) |
| Cocci patient | 0.811¹ | 1.142 | 1.416 | 0.213 | 0.518 |
| Histo patient | 0.102 | 0.683 | 0.088 | 0.127 | 1.240 |
| Normal subject | 0.056 | 0.040 | 0.024 | 0.018 | 0.025 |

A-C = 4 different rabbits, vaccinated with galactomannan (GM) or mould;
¹Data presented as optical density

As shown in Table 5, antibodies from rabbits immunized with Coccidioides antigens showed two patterns of recognition of Coccidioides and Histoplasma antigens: Pattern A recognized an antigen in the urine of the patient with coccidioidomycosis well but that from the patient with histoplasmosis poorly while pattern B recognized both well. Rabbit A was immunized with galactomannan vaccine and rabbit B was immunized with the mould vaccine.

TABLE 5

| Patient urine | Pattern A | Pattern B |
|---|---|---|
| Cocci patient | 2.28 | 2.13 |
| Histo patient | 0.09 | 0.68 |
| Normal subject | 0.02 | 0.04 |

### Example 11

### Evaluation of the Coccidioides Antigen Assay for Detection of Antigen in Patients

### Clinical and Laboratory Findings in Coccidioidomycosis Cases.

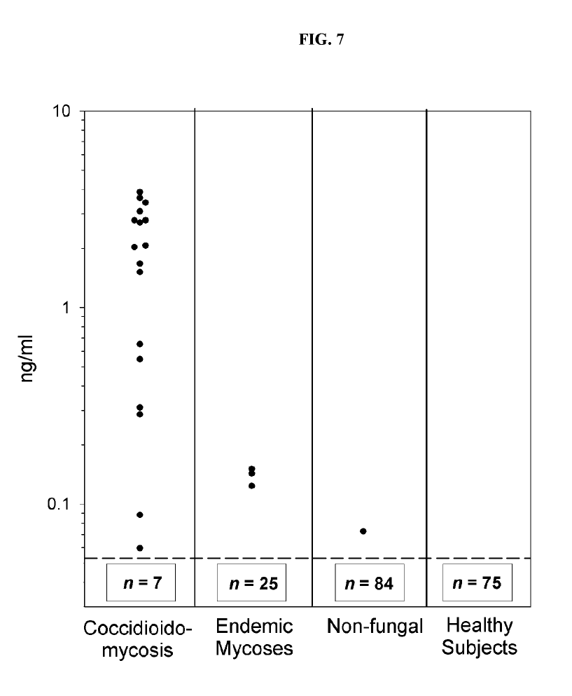

To evaluate the specificity and sensitivity of the Coccidioides EIA when compared with the Histoplasma EIA, samples from 24 patients with coccidioidomycosis and controls with other mycoses or non-fungal infections were tested in the two assays. Results from the study are shown in Table 6. Antigenuria was detected in 17 of 24 (70.8% [95% CI 50.8, 85.1%]) patients with coccidioidomycosis. Specificity was evaluated in patients without fungal infection, among whom antigen was detected in one of 160 controls (0.6% [95% CI 0.1, 3.4%]) (FIG. 7). Of 28 controls with other endemic mycoses, antigenuria was detected in three (10.7% [95% CI3.7, 27.2%]), two with histoplasmosis and one with paracoccidioidomycosis, at 0.12, 0.14 and 0.15 ng/mL, respectively. The Histoplasma antigen concentration was >39 ng/mL in two and 28.82 ng/mL in the third endemic control that was cross reactive in the Coccidioides EIA. Assuming a prevalence of coccidioidomycosis among patients tested for Coccidioides antigen of 5%, based upon experience at MiraVista Diagnostics, the positive predictive value is 85.7%, and the negative predictive value is 98.5% in patients with more severe forms of coccidioidomycosis.

The sensitivity for detection of antigenuria in patients with coccidioidomycosis was higher in the Coccidioides antigen EIA than in the Histoplasma antigen EIA. Antigenuria was detected in the Coccidioides EIA in 17 of 24 cases (70.8%) compared to 13 of 24 (54.2% in the Histoplasma antigen EIA. The highest sensitivity, 19 of 24 (79%), was achieved by combining the results both assays (table 6). Combined testing detected two patients were falsely negative in the Coccidioides assay. The first was patient 12 in table 6, an immunocompromised patient with pulmonary coccidioidomycosis. The second was patient 20, who was immunocompromised and had Coccidioides fungemia. Neither had positive cultures for H. capsulatum nor clinical findings suggestive for histoplasmosis.

TABLE 6

| | | | | | Antigen concentration- | |
|---|---|---|---|---|---|---|
| | Underlying | | Positive | | ng/mL | |
| No. | condition | Presentation | culture | Serology | Coccidioides | Histoplasma |
| 1 | Cirrhosis | Chronic | BAL | IgM +, IgG− | Negative | Negative |
| | Corticosteroids | pneumonia | | | | |
| | | Fatal | | | | |
| 2 | HIV | Acute | Bld, BAL, | IgM−, IgG− | 3.61 | 6.52 |
| | | pneumonia¹ | Ur | | | |
| 3 | HIV | Acute | BAL | IgM−, IgG+ | 0.38 | <0.6 |
| | | pneumonia | | | | |
| | | Fatal | | | | |
| 4 | Diabetes | Chronic | None | IgM +, IgG + | Negative | <0.6 |
| | | pneumonia¹ | | | | |
| 5 | Diabetes | Acute | Spt | IgM +, IgG + | 0.73 | Negative |
| | Corticosteroids | pneumonia | | | | |
| 6 | Pregnant | Chronic | None | CF 1:2 | Negative | Negative |
| | | pneumonia | | | | |
| 7 | HIV | Acute | Spt | IgM−, IgG− | 2.78 | 4.69 |
| | | pneumonia | | | | |
| 8 | None | Chronic | None | IgM +, IgG− | Negative | Negative |
| | | pneumonia | | | | |
| 9 | HIV | Acute | BAL, Spt, | IgM−, IgG−, CF− | 3.09 | 2.31 |
| | | pneumonia¹ | Bld | | | |
| | | Fatal | | | | |
| 10 | Cirrhosis | Acute | Spt | IgM+, IgG+, CF | 0.65 | 3.18 |
| | | pneumonia | | 1:32 | | |
| | | Fatal | | | | |
| 11 | HIV | Acute | BAL², Bld | IgM−, IgG−, CF − | 2.78 | 3.67 |
| | | pneumonia¹ | | | | |
| 12 | HIV | Acute | BAL² | IgM +, IgG + | 1.51 | Negative |
| | | pneumonia¹ | | | | |
| 13 | HIV | Acute | None | IgM +, IgG +, | Negative | Negative |
| | | pneumonia | | CF 1:16 | | |
| 14 | HIV | Acute | BAL² | IgM−, IgG−, CF− | 2.70 | Negative |
| | | pneumonia¹ | | | | |
| 15 | HIV | Acute | BAL | IgM+, IgG− | 0.09 | Negative |
| | | pneumonia | | | | |
| 16 | HIV | Acute | BAL², | CF 1:64 | 2.03 | <0.6 |
| | | pneumonia | Pleura | | | |
| 17 | Heart | Acute | Bld | IgM−, IgG− | 3.42 | 4.16 |
| | transplant | pneumonia¹ | | | | |
| | | Fatal | | | | |
| 18 | Kidney | Acute | Bld, BM, | IgM−, IgG−, CF | 3.88 | 1.23 |
| | transplant | pneumonia¹ | BAL | <1:2 | | |
| | | Fatal | | | | |
| 19 | None | Chronic | Soft tissue, | Not done | 0.55 | Negative |
| | | disseminated | bone | | | |
| 20 | HIV | Acute | Bld | CF 1:32 | Negative | 1.23 |
| | | pneumonia¹ | | | | |
| | | Fatal | | | | |
| 21 | HIV | Acute | BAL | IgM+, IGG+, CF | 0.31 | 2.66 |
| | | pneumonia | | 1:16 | | |
| 22 | HIV | Chronic | BAL² | Not done | 1.67 | <0.6 |
| | | pneumonia | | | | |
| 23 | None | Chronic | Soft tissue, | Not done | 2.07 | Negative |
| | | disseminated | bone | | | |
| 24 | HIV | Chronic | BAL² | Not done | Negative | Negative |
| | | pneumonia | | | | |

¹concurrent extrapulmonary,
²spherules seen by direct examination
Abbreviations:
HIV—human immunodeficiency virus,
BAL—bronchoalveolar lavage,
Bld—blood,
Ur—urine,
Spt—sputum,
BM—bone marrow,
IgM—immunoglobulin M,
IgG—immunoglobulin G,
CF—complement fixation

### Example 12

Detection of antigen in other body fluids. Patients with coccidioidomycosis often exhibit meningitis or infection of other serosal surfaces, such as the pleura, peritoneum, for synovium. Diagnosis often is made by isolating the organism from these surfaces. In overwhelming cases, the organism can be isolated from the blood. Test for detection of antigen in the blood or other body fluids could improve the sensitivity for diagnosis of coccidioidomycosis in such cases. Accordingly, the Coccidioides antigen EIA was evaluated for detection of antigen in other body fluids. Spike and recovery experiments were performed to assess the matrix effect of different body fluids on the results obtained with the Coccidioides antigen assay. Coccidioides galactomannan was added to serum, urine, bronchoalveolar lavage fluid, and cerebrospinal fluid (CSF) that were negative for Coccidioides antigen. Galactomannan concentration was measured in the Coccidioides antigen assay. The concentration in the spiked specimen was 1.60 ng/mL in CSF and 1.10 ng/mL in BAL and 1.33 ng/mL. In clinical testing between Jul. 1, 2008 and Mar. 23, 2009, positive results have been observed in 12 of 68 (17.3%) body fluids other than urine or serum, including CSF, bronchoalveolar lavage fluid, pleural fluid, synovial fluid, and abscess fluid.

Antigen detection in serum or plasma may be inhibited by antibodies present in the specimen. In the spike and recovery experiment, antigen was not detectable in normal serum. Presumably antibodies present in serum form immune complexes with circulating antigen, reducing the antigen's availability to attach to antibody coated plates. Pre-treatment of serum at 100° C. in the presence of ethylene diamine tetra acetic acid (EDTA) dissociates antigen-antibody complexes and denatures the freed antibody. To determine if EDTA-heat treatment would permit detection of antigenemia, 300 μL of spiked or negative serum and 100 μL 0.1 M EDTA were mixed and heated at 100° C. for 6 minutes, then centrifuged at 10,000×g for 10 minutes. The optical density of the negative control was 0.020, the untreated serum was 0.079, and the EDTA-heat treated serum was 0.845. Subsequently this approach was evaluated in clinical specimens. Antigen was detected in the serum in eight of 28 (28.6%) patients with coccidioidomycosis without EDTA-heat treatment compared to 19 of 28 (67.9%) following EDTA-heat treatment. These findings demonstrate that Coccidioides antigen may be detected in body fluids other than urine, and that EDTA-heat treatment improves the ability to detect Coccidioides antigenemia.

### Example 14

### Kit for Immunoassay and Associated Immunoassay Method

This example describes a kit for performing an immunoassay to detect a glycoprotein antigen circulating in the blood and excreted in the urine of patients with coccidioidomycosis. The kit can be used to perform a quantitative Coccidioides antigen EIA useful to diagnose coccidioidomycosis, monitor the response to therapy, and to diagnose relapse. Immunoglobulins with specific activity directed to Coccidioides antigen are employed to detect this antigen in patient samples. Purified immunoglobulin is bound to the surface of a microtiter plate. Coccidioides antigen in patient specimens will bind to the immunoglobulin and subsequently is then detected with a biotin-conjugated immunoglobulin digested to F(ab′)₂ specific to Coccidioides antigen followed by streptavidin-HRP and TMB substrate.

The kit preferably comprises the following components and reagents:
Prepared Microtiter Plates Coated with anti-Coccidioides antigen rabbit IgG and blocked with blocking agent having a coefficient of variability of less than about 0.2%, preferably substantially free of BSA (Starting Block-TBS (Thermo Scientific, Rockford, Ill.);
Conjugate Diluent, Blocking agent having a coefficient of variability of less than about 0.2%, preferably substantially free of BSA (e.g., Starting Block-TBS (Thermo Scientific Rockford, Ill.) used undiluted from the bottle for both the biotin and streptavidin conjugates)
Normal Rabbit Serum screened for desirably reducing interference with GARA;
Biotin-conjugated anti-Coccidioides Ag rabbit IgG, or the F(ab′)₂ or the F(ab) fragment of a anti-Coccidioides
Ag rabbit IgG. A working dilution can be made by diluting (for example, at 1:7280) in the conjugate diluent containing 5% normal rabbit serum screened for desirably reducing interference with GARA, as described above;
Streptavidin-HRP Conjugate: A working dilution is made by diluting 1:50,000 in the conjugate diluent;
TMB Substrate (e.g., a single component system);
EIA Wash Tablets (e.g., dissolve one tablet in 1 L of 18 MΩ lab quality water; wash is only good on the day it is prepared);
2.0 N Sulfuric Acid (Stop Solution);
Positive Controls (including High Positive Control and Low Positive Control);
Negative Control and Calibrator (Two negative controls may be supplied in the kit and used in each assay run as the cutoff calibrators; an additional negative control may also be used);
Quantitative Curve Standards (Nine standards supplied in the kit are run in each assay to plot the standard curve; the standards can include: 8.2, 5.9, 4.0, 1.9, 0.63, 0.31, 0.18, 0.07 and 0.00 ng/ml concentrations).

The kit may be used in combination with the following supplies and equipment:
ELISA plate reader;
Water purification system;
Vortex;
Laboratory Refrigerator;
37° C. Incubator;
Immuno plate washer with vacuum pressure station;
Single and Multichannel Pipettors with disposable tips;
Non sterile gloves;
Face shield or goggles;
Plate Sealer (provided in kit); and
A computer with software capable of conducting a 4 parameter curve analysis

Preferably, the assay controls meet the following criteria:
For the assay to be acceptable the controls must meet the following criteria:
   Mean negative calibrator controls must have an OD₄₅₀-OD₆₂₀<0.100.
   Negative control must be less than the calculated assay cutoff
   Low positive control must be 2.0 ng/ml±1.0 ng/ml.
   High positive control must be >8.2 ng/ml.
Do not use reagents beyond the expiration date
Any positive specimen will be repeated to confirm the positive result.

The kit may include instruction for use comprising the following procedure:
1. All specimens are handled following universal precautions.
2. Remove appropriate number of precoated plates/wells from the refrigerator. Plates have removable strips of wells. All wells not being used should be removed from the plate and returned to storage pouch and placed back in the refrigerator. Do not remove the desiccant pouch from the plate storage bag.
3. Allow components to come to room temperature (approximately 20 minutes).
4. Add 100 μl/well of the control or specimen to be tested. All samples are tested in the following order on each plate: Negative calibrator control 1, negative calibrator control 2, high positive control, low positive control, negative control, quantitative curve standards, patient samples.
5. Seal each plate and incubate at 37° C. for 1 hour.
6. Wash the plate 5× with freshly prepared EIA wash using an Immuno plate washer.
7. Prepare a 5% solution of NRS in conjugate diluent, and prepare a 1:7,280 dilution of biotin-conjugate into that solution, and add 100 μl/well to the microtiter plate.
8. Reseal each plate and incubate at 37° C. for 1 hour.
9. Wash the plate 5× with EIA wash as in step 6 above.
10. Prepare a 1:50,000 dilution of HRP labeled streptavidin in the conjugate diluent by first preparing a 1:1000 dilution (1 μL in 1 mL) and from this make a 1:50 dilution for a final 1:50,000 dilution. Add 100 μl/well to the microtiter plate.
11. Reseal each plate and incubate at 37° C. for 1 hour.
12. Wash the plate 5× with EIA wash as described in step 6.
13. Add 100 μl/well of TMB Peroxidase Substrate that has been brought to room temperature. Develop the plate for 12 minutes at room temperature without a plate sealer. Do not place the plate in direct light during the development time.
14. Stop the reaction by adding 100 μl/well of 2.0 N sulfuric acid stop solution.
15. Color development is measured by reading the optical density on the EIA Plate reader at OD₄₅₀-OD₆₂₀. The plate should be read within 30 minutes of adding the stop solution.

To calculate the assay cutoff the following equation may be used: Mean OD of negative calibrator controls×Multiplier (M)=Cutoff, where M=2.0 if the mean of the negative calibrator controls is >0.050 and M=3.0 if the mean of the negative calibrator controls is <0.050. Preferably, the negative control is less than the cutoff To plot the calibration curve, the following 4 parameter formula may be used:

$$y = min + \frac{max - min}{1 + {( x / EC 50 )}^{Hillslope}}$$

The controls are preferably selected to meet the following criteria: Low positive control that is ng/ml and a High positive control must be ≧ng/ml. Preferably, the R² value for the line is ≧0.98.

Using the calibration curve, patient results are preferably determined by calculating from the standard curve. Patients with results higher than the highest standard can be reported Patients with results less than the cutoff can be reported as “none detected.” The reportable range is <0.076-8.2 ng/ml. Results of none detected are negative. Results above the cutoff are positive and interpreted using the following guideline.

| Specimen Result | | Result Interpretation |
|---|---|---|
| None Detected | | Negative |
| 0.07-0.5 | ng/ml | Positive, low |
| 0.51-2.0 | ng/ml | Positive, moderate |
| >2.0 | ng/ml | Positive, high |

The examples above illustrate the invention, but are not to be taken as limiting the various aspects of the invention so illustrated.

What is claimed:

1. A method for the detection of a Coccidiodes antigen comprising the steps of:
a. providing a binding surface;
b. contacting the binding surface with a capture antibody selected from an anti-Coccidioides antibody or an anti-Coccidioides antibody fragment;
c. blocking the binding surface with a blocking agent to yield an antigen binding surface;
d. contacting the antigen binding surface with an analyte in a manner effective to bind an Coccidioides antigen to the antigen binding surface;
e. contacting the bound antigen with a detector antibody selected from an anti-Coccidioides antibody or an anti-Coccidioides antibody fragment; and
f. detecting the bound antigen.

2. The method of claim 1 wherein the detector antibody is an anti-Coccidioides IgG antibody, F(ab′)₂ fragment of an anti-Coccidioides IgG antibody or F(ab) fragment of an anti-Coccidioides IgG antibody.

3. The method of claim 1, wherein the capture antibody is a anti-Coccidioides IgG antibody, F(ab′)₂ fragment of an anti-Coccidioides IgG antibody or F(ab) fragment of an anti-Coccidioides IgG antibody.

4. The method of claim 1 wherein the capture antibody and detector antibody are each independently chosen from the group consisting of a rabbit anti-Coccidioides IgG antibody, a F(ab′)₂ fragment of a rabbit anti-Coccidioides IgG antibody and a F(ab) fragment of a rabbit anti-Coccidioides IgG antibody.

5. The method of claim 4 wherein the capture antibody and the detector antibody are each a rabbit anti-Coccidioides IgG antibody.

6. The method of claim 4 wherein the detector antibody is an anti-Coccidioides antibody generated by immunizing a rabbit with a vaccine comprising Coccidioides.

7. The method of claim 4 wherein the detector antibody is an anti-Coccidioides antibody generated by immunizing a rabbit with a vaccine comprising galactomannan purified from Coccidioides.

8. The method of claim 4 wherein the capture antibody is an anti-Coccidioides antibody generated by immunizing a rabbit with a vaccine comprising Coccidioides.

9. The method of claim 4 wherein the capture antibody is an anti-Coccidioides antibody generated by immunizing a rabbit with a vaccine comprising galactomannan purified from Coccidioides.

10. The method of claim 4 wherein the detector antibody is an anti-Coccidioides antibody generated by immunizing a rabbit with a vaccine comprising galactomannan purified from Coccidioides and the capture antibody is an anti-Coccidioides antibody generated by immunizing a rabbit vaccine comprising Coccidioides.

11. The method of claim 10 wherein the detector and the capture antibody are IgG antibodies.

12. The method of claim 10, wherein the detector antibody is a F(ab′)₂ and the capture antibody is an IgG.

13. The method of claim 4 wherein the blocking agent is Starting Block™.

14. The method of claim 4 further comprising: combining the detector antibody with a normal rabbit serum before contacting the detector antibody with the bound antigen.

15. The method of claim 4, further comprising the steps of:
a. selecting a normal rabbit serum based on a serum screening assay comprising the steps of:
i. providing a normal rabbit serum sample,
ii. performing a screening test assay to measure the detected level of binding of a rabbit anti-Coccidioides IgG detector antibody to a rabbit anti-Coccidioides IgG capture antibody in the presence of goat anti-rabbit and the serum sample;
iii. performing a control test assay to measure the detected level of binding of a rabbit anti-Coccidioides IgG detector antibody to the rabbit anti-Coccidioides IgG capture antibody in the absence of goat anti-rabbit antibody and in the presence of the serum sample;
iv. selecting the serum sample if level of binding of the detector antibody to the capture antibody in the screening assay is less than 3 times the level of binding of the detector antibody to the capture antibody in the control test assay; and
b. combining the detector antibody with the normal rabbit serum selected in step (a) prior to contacting the detector antibody with the bound antigen.

16. The method of claim 4, wherein the detector antibody is conjugated to biotin.

17. The method of claim 17 further comprising the steps of:
a. contacting the detector antibody comprising with horseradish peroxidase labeled streptavidin under conditions that allow for streptavidin binding to biotin;
b. contacting the bound horseradish peroxidase with tetramethylbenzidine in a manner effective to convert the tetramethylbenzidine to a detectable chromophore; and
c. detecting the presence of the detector antibody by measuring the optical density of the chromophore at two or more wavelengths.

18. The method of claim 4, wherein the analyte comprises blood serum, urine, cerebrospinal fluid, bronchoalveolar lavage fluid, pleural fluid, pericardial fluid, peritoneal fluid, synovial fluid, ocular fluid, and abscess contents.

19. A kit for detection of an antigen in an analyte, the kit comprising:
a. a means for capturing an antigen in the analyte to form a bound antigen;
b. a detection antibody composition adapted to bind to the bound antigen; and
c. a means for detecting the detection antibody bound to the antigen wherein the kit comprises one or more components selected from the group consisting of:
i. the detector antibody composition comprising a modified polyclonal rabbit anti-Coccidioides IgG antibody,
ii. the detector antibody composition further comprising a normal rabbit serum that minimizes the detected level of binding of the detector antibody to the capture antibody in the presence of goat anti-rabbit antibody.
